(19) United States
(12) Patent Application Publication
(10) Pub. No.: US 2006/0257499 A1
(43) Pub. Date: Nov. 16, 2006

(54) Vaginal rinse with enhanced bacteriocidal activity

(76) Inventors: Leonard Mackles, New York, NY (US); William S. Bess, Edison, NJ (US)

Correspondence Address: OMRI M. BEHR, 325 PIERSON AVENUE, EDISON, NJ, 08837-3123, US

(21) Appl. No.: 11/449,288

(22) Filed: Jun. 8, 2006

Related U.S. Application Data
(63) Continuation-in-part of application No. 11/019,485, filed on Dec. 22, 2004.

(51) Int. Cl. A61K 33/40; A61K 33/26; A61K 33/06; A61K 33/34; A61K 33/32; A61K 31/17; A61K 33/00

(52) U.S. Cl. 424/616; 424/630; 424/641; 424/646; 424/722; 424/682; 514/588

(57) ABSTRACT

There are provided vaginal rinse compositions, consisting essentially of: a) 1-20 wt % of a mucosally acceptable peroxide, b) 0.5-15 wt % of a mucosally acceptable sulfate, bisulfate, pyrosulfate salt of an inorganic cation or mixtures thereof, and c) water to 100 wt %. There are further provided methods of preparing and using said compositions as well as kits for maintaining the components to prepare said compositions.

### RELATED APPLICATIONS

This application is a continuation in part of our application Ser. No. 11/019,485 filed Dec. 22, 2004.

### FIELD OF THE INVENTION

Improved vaginal product compositions.

### BACKGROUND OF THE INVENTION

Vaginal compositions have been used for the prevention of bad odors, elimination of microorganisms that are responsible for these odors, low level vaginal infections, lubrication, as antifungal treatments, and as moisturizers. Vaginal rinses containing hydrogen peroxide are well known for their ability to reduce the bacterial flora in the cavity. Hydrogen peroxide is utilized due to its ability to decompose into water and oxygen, with the oxygen then acting as an antimicrobial agent. It has been noted that pre-heated or warmed compositions are more comfortable to use and may provide additional benefits.

Day U.S. Pat. No. 6,692,757 discloses a system for cleaning water lines, particularly in dental offices in which the peroxide decomposition is accelerated by the presence of an acidic sulfate. The presence of a disinfectant is also required. There is no disclosure or suggestion of use.

A heated vaginal rinse will kill microbes to a greater extent than an unheated vaginal rinse of the same composition. Higher temperature will accelerate the efficacy of an antimicrobial or antifungal agent

The generation of heat in solutions for hair bleaching and dying using a combination of hydrogen peroxide and sulfites is well known, but there has been no disclosure of suggestion of such combinations in the vaginal cavity.

The parent application of this application discloses that the compositions herein are useful as oral rinses and for tooth whitening. We have found that these compositions also find use as vaginal products.

### SUMMARY OF THE INVENTION

The present invention provides a novel group of vaginal products which are pre-warmed thus providing more comfort in use. Additionally, antimicrobial and/or antifungal efficacy will be enhanced in vaginal products containing those agents

One embodiment utilizes an in-situ formation of sulfate, bisulfate or pyrosulfate or mixture thereof from the reaction between sulfites, bisulfites or metabisulfites. This embodiment suitably utilizes a 2-phase system that is mixed just prior to use in the cavity. Part 1 of the system contains a mucosally acceptable inorganic cation salt of sulfite, bisulfite, metabisulfite or mixture thereof. Part 2 contains a mucosally acceptable peroxide in an amount sufficient to convert the sulfite, bisulfite, metabisulfite or mixture thereof. Other ingredients such as flavor, lubricants etc. may optionally be included when desired, as described below.

A particularly preferred embodiment utilizes an in-situ formation of sulfate, bisulfate pyrosulfate or mixture thereof from the reaction between sulfites, bisulfites or metabisulfites and peroxide to form sulfates. This embodiment suitably utilizes a 2-phase system that is mixed just prior to use in the cavity. Part 1 of the system contains a mucosally acceptable inorganic cation salt of sulfite, bisulfite, metabisulfite or mixture thereof. Part 2 contains a mucosally acceptable peroxide in an amount that exceeds the stoichiometric amount required to convert the sulfite, bisulfite, metabisulfite or mixture thereof. While it is generally preferred to employ a small excess of peroxide, say about a 0.5% excess, the invention is not limited thereto. Indeed, in certain uses an excess of the charged inorganic cation salt may be desirable. Other ingredients such as lubricants, antifungal agents, additional antimicrobial agents, anti-itch agents, humectants, colors, fragrances, etc. and surfactant in an amount to enable solubilization of the latter. may optionally be included when desired, as described below.

During the reaction, not only is a sulfate and or a bisulfate formed but also the reaction is exothermic, which results in a temperature increase of about 3 to about 30 degrees centigrade depending on the concentrations employed. The resulting mixture forms the vaginal product, which if used substantially at once after formation, increased temperature, which will increase the rate of peroxide decomposition, and result in greater antimicrobial efficacy.

### DESCRIPTION OF THE PREFERRED EMBODIMENTS

For the reaction creating the oxidized salt to be sufficiently exothermic, sulfite, bisulfite or metabisulfite initial levels at the higher end of this range are preferred. Clearly there needs to be sufficient peroxide to generate the exothermic reaction. If desired, a sufficient excess of peroxide can be included to generate the active oxygen provided by the acceleration of decomposition caused by the rise in temperature This excess may be 0.5 wt % of the solution used, but suitably may rise to about 5 wt %, though this should not be considered a limiting amount.

Thus the vaginal product composition which is utilized suitably consists essentially of: a) 1-20 wt % of a mucosally acceptable peroxide, b) 0.5-15 wt % of a mucosally acceptable sulfate, bisulfate, pyrosulfate salt of an inorganic cation or mixtures thereof, and c) water to 100 wt %. This may be prepared by mixing i) an aqueous carrier, which may be a solution or water containing emulsion consisting essentially of 1-40 wt % of the peroxide, and ii) an aqueous aqueous carrier, which may be a solution or water containing emulsion consisting essentially of 0.5-30 wt % of the said inorganic cation salt of a sulfite, bisulfite, metabisulfite or mixture thereof. Optionally it may provide that the amount of peroxide in (i) exceeds the stoichiometric amount required to completely oxidize the salt of the sulfite, bisulfite, metabisulfite or mixture thereof, by at least 0.5 wt %. Desirably, the components i) and ii) are contained in a kit of containers each containing one of these components.

Suitably, the peroxide is any mucosally acceptable peroxide, suitably hydrogen peroxide, urea peroxide or mixtures thereof and the cation is selected from the group consisting of sodium, potassium, ammonium, magnesium, calcium, aluminum, zinc, iron or mixture thereof.

The product may contain other ingredients conventionally used in vaginal products. These include but are not limited to lubricants, antifungal agents, additional antimicrobial agents, anti-itch agents, humectants, colors, fragrances, etc. and surfactant in an amount to enable solubilization of the latter.

While in no way to be considered as limiting, favorable ranges in the present invention of peroxide/bisulfate vaginal products consist essentially of from about 1.5 to about 6 wt %, preferably about 3 wt % peroxide and from about 3 to about 10 wt %, preferably about 5 wt % bisulfate. Additionally the products may contain lubricants, antifungal agents, additional antimicrobial agents, anti-itch agents, humectants, colors, fragrances, etc. and surfactant in an amount to enable solubilization of the latter.

### EXAMPLES

### Example 1

### Vaginal Cleanser with Excess Peroxide

| Ingredient | % by Weight |
|---|---|
| Phase A | |
| Water | 96.15 |
| Fragrance | 0.10 |
| Benzoic Acid | 0.25 |
| Cocamidopropyl Betaine | 0.50 |
| Sodium Bisulfite | 3.00 |
| Total | 100.00 |
| Phase B | |
| Water | 98.38 |
| Hydrogen Peroxide (50%) | 5.00 |
| Total | 100.00 |

Phase A: Combine Water and Benzoic Acid.
   While mixing, slowly add the Betaine
   Mix until clear.
Phase B: Combine ingredients and mix until uniform

### Example 2

### Vaginal Lubricant

| Ingredient | % by Weight |
|---|---|
| Phase A | |
| Glycerin | 25.00 |
| Fragrance | 0.20 |
| Benzoic Acid | 0.25 |
| Cocamidopropyl Betaine | 1.00 |
| Water | 70.55 |
| Sodium Bisulfite | 2.50 |
| Hydroxyethylcellulose | 0.50 |
| Total | 100.00 |
| Phase B | |
| Water | 98.65 |
| Hydrogen Peroxide (50%) | 1.35 |
| Total | 100.00 |

Phase A: Mix hydroxyethylcellulose and water until uniform. Add remaining ingredients and mix until uniform.
Phase B: Combine ingredients and mix until uniform

### Example 3

### Antifungal Vaginal Cream

| Ingredient | % by Weight |
|---|---|
| Phase A: | |
| Mineral Oil 80-90 SUS | 10.00 |
| Fragrance | 0.20 |
| Glyceryl Monostearate SE | 5.00 |
| Glycerin | 5.00 |
| Water | 73.80 |
| Sodium Bisulfite | 4.00 |
| Miconozole Nitrate | 2.00 |
| Total | 100.00 |
| Phase B: | |
| Hydrogen Peroxide(50% Aq. Sol'n) | 2.16 |
| Water | 82.84 |
| Mineral Oil 80-90 SUS | 10.00 |
| Glyceryl Monostearate SE | 5.00 |
| Total | 100.00 |

Phase A: Combine Water, Sodium Bisulfite, and Glycerin. Mix until clear While heating to 150° F. In a separate container combine mineral oil and glyceryl monostearate and heat to 150° F. While mixing combine add the oils to the water phase and mix while cooling to 85° F. Add the Miconozole Nitrate and mix until uniform.

Phase B: Heat the water to 150° F. In a separate container combine the Mineral Oil and glyceryl monostearate and heat to 150° F. While mixing combine add the oils to the water phase and mix while cooling to 85° F. Add the hydrogen peroxide and mix until uniform.

In accordance with the above formulation, but where in place of sulfite and/or the metabisulfite there is utilized only the metabisulfite, sulfite, or bisulfite, a similar solution is obtained.

In accordance with the above formulations, but where in place of sodium the cation utilized is potassium, ammonium, magnesium, calcium, aluminum, zinc, iron or mixture thereof a similar formulation is obtained.

Similarly in place of hydrogen peroxide, there may be utilized urea peroxide or mixtures thereof with hydrogen peroxide.

The formulation is utilized by introducing a comfortable amount into the vagina.

We Claim:

1. An vaginal composition,
consisting essentially of:
a) 1-20 wt % of a mucosally acceptable peroxide
b) 0.5-15 wt % of a mucosally acceptable sulfate, bisulfate, pyrosulfate salt of an inorganic cation or mixtures thereof,
c) water to 100 wt %
which is prepared by mixing
i) an aqueous carrier consisting essentially of 1-40 wt % of the peroxide, and
ii) an aqueous carrier consisting essentially of 0.5-30 wt % of the said inorganic cation salt of a sulfite, bisulfite, metabisulfite or mixture thereof.

2. The composition of claim 1 wherein the final amount of salt is between about 1.5 and about 6.0 wt %.

3. The composition of claim 1 where the peroxide is hydrogen peroxide, urea peroxide or mixtures thereof.

4. The composition of claim 2 where the peroxide is hydrogen peroxide, urea peroxide or mixtures thereof.

5. The composition of claim 3 where the cation is selected from the group consisting of sodium, potassium, ammonium, magnesium, calcium, aluminum, zinc, iron or mixture thereof.

6. The composition of claim 4 where the cation is selected from the group consisting of sodium, potassium, ammonium, magnesium, calcium, aluminum, zinc, iron or mixture thereof.

7. A method of preparing an vaginal composition, consisting essentially of:
a) 1-20 wt % of a mucosally acceptable peroxide
b) 0.5-15 wt % of a mucosally acceptable sulfate, bisulfate, pyrosulfate salt of an inorganic cation or mixtures thereof,
c) water to 100 wt %
which comprises mixing
i) an aqueous carrier consisting essentially of 1-40 wt % of said acceptable peroxide and
ii) an aqueous carrier consisting essentially of 0.5-30 wt % of said inorganic cation salt of a sulfite, bisulfite, metabisulfite or mixture thereof,

8. The method of claim 7 wherein the final amount of salt is between about 1.5 and about 6.0 wt %.

9. The method of claim 7 where the peroxide is hydrogen peroxide, urea peroxide or mixtures thereof.

10. The method of claim 8 where the peroxide is hydrogen peroxide, urea peroxide or mixtures thereof.

11. The method of claim 9 where the cation is selected from the group consisting of sodium, potassium, ammonium, magnesium, calcium, aluminum, zinc, iron or mixture thereof.

12. The method of claim 10 where the cation is selected from the group consisting of sodium, potassium, ammonium, magnesium, calcium, aluminum, zinc, iron or mixture thereof.

13. A method of enhancing the bacteriocidal activity of peroxide in the vaginal cavity comprising the steps of:
mixing aqueous carriers consisting essentially of
i) an aqueous carrier consisting essentially of 1-40 wt % of a mucosally acceptable peroxide and
ii) an aqueous carrier consisting essentially of 0.5-30 wt % of an inorganic cation salt of a sulfite, bisulfite, metabisulfite or mixture thereof, and introducing said carrier into the cavity.

14. The method of claim 13 wherein the mixture is introduced into the cavity promptly after its production.

15. The method of claim 13 wherein the final amount of salt is between about 1.5 and about 6.0 wt %.

16. The method of claim 13 where the peroxide is hydrogen peroxide, urea peroxide or mixtures thereof.

17. The method of claim 15 where the peroxide is hydrogen peroxide, urea peroxide or mixtures thereof.

18. The method of claim 16 where the cation is selected from the group consisting of sodium, potassium, ammonium, magnesium, calcium, aluminum, zinc, iron or mixture thereof.

19. The method of claim 17 where the cation is selected from the group consisting of sodium, potassium, ammonium, magnesium, calcium, aluminum, zinc, iron or mixture thereof.

20. A kit for preparing a raising internal vaginal temperature having bacteriocidal action in the vaginal cavity consisting essentially of
i) a container containing an aqueous carrier consisting essentially of 1-40 wt % of a mucosally acceptable peroxide and
ii) a container containing an aqueous carrier consisting essentially of 0.5-30 wt % of an inorganic cation salt of a sulfite, bisulfite, metabisulfite or mixture thereof.

21. The kit of claim 20 wherein the final amount of salt is between about 1.5 and about 6.0 wt %.

22. The kit of claim 20 where the peroxide is hydrogen peroxide, urea peroxide or mixtures thereof.

23. The kit of claim 21 where the peroxide is hydrogen peroxide, urea peroxide or mixtures thereof.

24. The kit of claim 22 where the cation is selected from the group consisting of sodium, potassium, ammonium, magnesium, calcium, aluminum, zinc, iron or mixture thereof.

25. The kit of claim 23 where the cation is selected from the group consisting of sodium, potassium, ammonium, magnesium, calcium, aluminum, zinc, iron or mixture thereof.

26. A method of raising the internal temperature of the vaginal cavity by introducing thereinto one member of one of the two groups set forth herein consisting essentially of:
i) an aqueous carrier consisting essentially of 1-40 wt % of a mucosally acceptable peroxide and
ii) an aqueous carrier consisting essentially of 0.5-30 wt % of an inorganic cation salt of a sulfite, bisulfite, metabisulfite or mixture thereof,
and thereafter introducing into said cavity a member of the other group.
